(19) United States
(12) Patent Application Publication
(10) Pub. No.: US 2022/0273582 A1
(43) Pub. Date: Sep. 1, 2022

(54) CONTINUOUS METHOD FOR NANO-EMULSIFICATION BY CONCENTRATION PHASE INVERSION

(71) Applicants: UNIVERSITÉ D'ANGERS, Angers (FR); INSERM (INSTITUT NATIONAL DE LA SANTÉ ET DE LA RECHERCHE MÉDICALE), Paris Cedex 13 (FR); CENTRE NATIONAL DE LA RECHERCHE SCIENTIFIQUE, Paris (FR)

(72) Inventors: Brice CALVIGNAC, Trelaze (FR); Jean-Christophe GIMEL, Angers (FR); Emilie ROGER, Angers (FR); Nicolas ROLLEY, Saint-Barthélemy-d'Anjou (FR); Guillaume LEFEBVRE, Angers (FR); Guillaume BASTIAT, Briollay (FR); Florian FOUCHET, Écouflant (FR); Kévin MATHA, Angers (FR)

(73) Assignees: UNIVERSITÉ D'ANGERS, Angers (FR); INSERM (INSTITUT NATIONAL DE LA SANTÉ ET DE LA RECHERCHE MÉDICALE), Paris Cedex 13 (FR); CENTRE NATIONAL DE LA RECHERCHE SCIENTIFIQUE, Paris (FR)

(21) Appl. No.: 17/626,914

(22) PCT Filed: Jul. 24, 2020
(86) PCT No.: PCT/FR2020/051365

§ 371 Date: Jan. 13, 2022

(30) Foreign Application Priority Data
Jul. 24, 2019 (FR) FR1908392

(51) Int. Cl. A61K 9/51; A61K 9/107; A61K 45/06

(52) U.S. Cl. CPC A61K 9/5192; A61K 9/1075; A61K 9/5123; A61K 45/06; B82Y 5/00

(57) ABSTRACT

A continuous process for nano-emulsification that is performed by concentration phase inversion in a microfluidic reactor, including the following steps: (a) injection of an aqueous phase into a first microchannel, the first microchannel opening onto a formulation chamber, (b) injection, into a second microchannel, of a fatty phase including one or more fatty substances immiscible in the aqueous phase, and one or more surfactants, the second microchannel opening into the formulation chamber, then (c) mixing of the aqueous phase and the fatty phase in the formulation chamber, then (d) recovering, at the output of the formulation chamber, a suspension including lipid nanocapsules. Also, the lipid nanocapsules obtainable by the process, and the use of the lipid nanocapsules as nanovectors for pharmacologically active ingredients.

FIELD OF INVENTION

The present invention relates to the field of nanoemulsion formulations, more particularly the invention relates to a continuous process for nano-emulsification carried out by concentration phase inversion (CPI).

The present invention also relates to lipid nanocapsules obtainable by the process according to the invention.

Finally, the present invention relates to the use of the lipid nanocapsules according to the invention for the encapsulation of molecules such as a pharmacologically active molecule.

### BACKGROUND OF INVENTION

Nanoformulations, such as lipid nanoemulsions (LNE), solid lipid nanoparticles (SLN) or even nanostructured lipid vectors such as lipid nanocapsules (LNC) (Matougui et al., 2016, Int. J. Pharm., 502, 80-97) have been of increasing interest in recent years, in particular in the pharmaceutical, cosmetic and food industries.

The kinetically very stable lipid nanocapsules are not very sensitive to changes in temperature and in composition. They are of very particular interest. For example, it has been shown that these nanoformulations could be used as encapsulation and drug delivery systems (Hörmann and Zimmer, 2016, J. Controlled Release, 223, 85-98).

Two main techniques are used for the production of lipid nanocapsules:
High energy methods, such as high-pressure homogenization (HPH) technology and ultrasound technology.
Low energy methods, including emulsions phase inversion processes by thermal effect, by change in composition or also spontaneous methods.

Since high-energy methods are particularly energy-consuming, they may not be recommended for the encapsulation of thermo- and/or mechano-sensitive molecules, such as proteins or peptides. Technologies that consume less energy and use milder formulation conditions should therefore be preferred.

Patent WO2001064328 describes a process for formulating lipid nanocapsules by temperature phase inversion, “TPI process”. However, since this process is based on a temperature variation over time, it does not either allow the use of heat-sensitive molecules.

The document Lefebvre et al., Int. J. of Pharm., 534 (1-2), 2017 discloses a “batch” process for preparing lipid nanocapsules by concentration phase inversion “CPI process”. It consists of the formation of an oily phase (surfactant and co-surfactant dispersed in oil) to which all of the water will then be added. The possibility with the CPI process of reducing the formulation temperature up to 20° C. (compared to 70° C.-90° C. by the TPI process depending on the NaCl concentration) has been shown. However, a risk of the described method lies in the difficulty of controlling the operating conditions (temperature and mixing conditions) and variabilities in the size of the lipid nanocapsules as well as in their size polydispersity index (known as PDI, Polydispersity Index) are often observed.

Thus, with a view of producing lipid nanocapsules of uniform size and exhibiting suitable polydispersity for optimum efficiency for a given application (for example cell internalization and crossing of biological barriers), there is a need for a process for formulating lipid nanocapsules allowing control of the operating conditions. It would be also interesting to be able to envisage a process other than “batch” such as the one described, with a view to continuous production on an industrial scale.

It has been discovered and implemented a continuous nano-emulsification process characterized in that said process is carried out by concentration phase inversion (CPI) in a microfluidic reactor, which is the subject-matter of the present invention.

The process according to the invention has the advantage of providing lipid nanocapsules having a homogeneous and controlled particle size, that is to say with a very low polydispersity. Thus, the process according to the invention makes it possible in particular to produce lipid nanocapsules at different scales. Indeed, unlike a “batch” process, the continuous process according to the invention can be easily transposed to an industrial scale, for example by simply placing different microfluidic reactors in parallel or by using static mixers.

Another advantage of the process of the present invention is to be able to formulate, on demand, nanomedicines at low temperature, for example at body temperature, on an industrial scale for a production of nanomedicines on a large scale or on a laboratory scale for the production of personalized treatment.

### SUMMARY

The invention therefore relates to a continuous process for nano-emulsification wherein said process is carried out by concentration phase inversion (CPI) in a microfluidic reactor, and comprising the following steps:
a. injection into a first microchannel of an aqueous phase, said first microchannel opening into a formulation chamber,
b. injection into a second microchannel of a fatty phase comprising one or more fatty substances, and one or more surfactants, said second microchannel opening into said formulation chamber, then
c. mixing of the aqueous phase and the fatty phase in said formulation chamber, then
d. recovery, at the outlet of the formulation chamber, of a suspension comprising lipid nanocapsules.

In one embodiment, the surfactant(s) are chosen from nonionic hydrophilic surfactants, and mixtures thereof. In one embodiment, the surfactant(s) are chosen from mono- and di-esters of fatty acid and of polyethylene glycol, and mixtures thereof. In one embodiment, the surfactant(s) are chosen from mono- and di-esters of stearic acid and of polyethylene glycol, and mixtures thereof.

In one embodiment, the fatty substance(s) are chosen from glycerol mono-esters, di-esters and tri-esters, polyethylene glycol mono-esters and di-esters, and mixtures thereof. In one embodiment, the fatty substance(s) are chosen from C₈-C₁₈ triglycerides, and mixtures thereof. In one embodiment, the fatty substance(s) are chosen from capric and caprylic acids triglycerides and their mixtures thereof.

In one embodiment, the fatty phase further comprises one or more co-surfactants. In one embodiment, the fatty phase further comprises one or more co-surfactants chosen from nonionic surfactants. In one embodiment, the fatty phase further comprises one or more co-surfactants chosen from sorbitan monooleate or diethylene glycol mono-ethyl ether, and mixtures thereof.

In one embodiment, the weight ratio of the sum of the flow rates of surfactants and co-surfactants to the flow rate of fatty substances in the formulation chamber is between 0.8 and 4. In one embodiment, the weight ratio of the sum of the flow rates of surfactants and co-surfactants to the flow rate of fatty substances in the formulation chamber is between 2 and 4.

In one embodiment, the weight ratio of the sum of the flow rates of surfactant, co-surfactants and fatty substances to the flow rate of the aqueous phase in the formulation chamber is between 0.03 and 0.3. In one embodiment, the weight ratio of the sum of the flow rates of surfactant, co-surfactants and fatty substances to the flow rate of the aqueous phase in the formulation chamber is between 0.04 and 0.2.

In one embodiment, the fatty phase further comprises water, in a content of between 0% and 30% by weight, relative to the total weight of the fatty phase. In one embodiment, the fatty phase further comprises water, in a content of between 0% and 20% by weight, relative to the total weight of the fatty phase. In one embodiment, the fatty phase further comprises water, in a content of between 0% and 15% by weight, relative to the total weight of the fatty phase.

In one embodiment, the first microchannel is thermalized at a temperature between 20° C. and 70° C., preferably between 30° C. and 50° C.

In one embodiment, the second microchannel is thermalized at a temperature between 20° C. and 70° C., preferably between 30° C. and 50° C.

The invention also relates to lipid nanocapsules obtainable by the process as described above, said nanocapsules comprising one or more co-surfactants chosen from nonionic surfactants, preferably chosen from sorbitan monooleate or diethylene glycol mono-ethyl ether and mixtures thereof.

In one embodiment, the lipid nanocapsules further comprise a heat-sensitive pharmacologically active ingredient, preferably chosen from peptides, proteins or nucleic acids, anticancer agents, anti-infective agents or antibiotics.

In one embodiment, the lipid nanocapsules have a particle size of between 20 and 100 nm, preferably between 15 and 50 nm, more preferably between 20 and 35 nm.

In one embodiment, the lipid nanocapsules have a polydispersity index of between 0.05 and 0.2, preferably between 0.05 and 0.1.

The invention also relates to the use of lipid nanocapsules as described above as nanovectors of pharmacologically active ingredient.

In one embodiment, the pharmacologically active ingredient is a heat-sensitive active.

### DEFINITIONS

In the present invention, the following terms have the following meanings:
“Active agent” relates to a compound of therapeutic or cosmetic interest. In one embodiment the active agent is a pharmacologically active molecule. In one embodiment, the active agent is a cosmetic active.
“Cosmetic active” relates to a substance or a mixture intended to be brought into contact with the superficial parts of the human body or with the teeth and the oral mucous membranes, with a view, exclusively or mainly, to clean them, to perfume them, to change their appearance, to protect them, to keep them in good condition or to correct body odors.
“Fatty substance”: designates a compound such as oils, lipids, lipophilic molecules and other non-polar solvents, capable of dissolving in fatty phases, but immiscible in aqueous phases at 25° C. and atmospheric pressure.
“Mixing chamber”: refers to the place where fluids of the same type are prepared. In one embodiment, the aqueous phase is prepared in a first mixing chamber. In one embodiment, the fatty phase is prepared in a second mixing chamber by mixing a fatty substance as defined in the present invention and a surfactant as defined in the present invention. In one embodiment, the mixing chamber in which the aqueous phase is prepared is connected via a first microchannel to a formulation chamber. In one embodiment, the mixing chamber in which the fatty phase is prepared is connected via a second microchannel to said formulation chamber.
“Formulation chamber”: refers to the place where said fatty phase and said aqueous phase are brought into contact in order to cause the nano-emulsification process, leading to the formation of the lipid nanocapsules according to the invention.
“Hydrophilic”: relates to a molecule or portion of molecule being negatively or positively charged or neutral, capable of forming hydrogen bonds, allowing easier dissolution in water than in oil or other solvents.
“Polydispersity index” or “PDI”: designates in the case of a single-mode size distribution, the ratio of the variance of the size of the particles to the square of the mean size of the particles.
“Lipophilic”: concerns a chemical compound capable of dissolving in fatty phases such as oil, lipids and other non-polar solvents.
“Microchannel”: relates to a channel whose characteristic dimension allows the flow of fluids such as liquids or gases. The microchannel can be delimited by a lower wall, an upper wall and two opposite side walls; the distance between the opposing side walls is the characteristic distance. In one embodiment, the microchannel has a characteristic distance of between about 100 μm and about 2000 μm. In one embodiment, the microchannel has a characteristic distance between 100 μm and 1500 μm. In one embodiment, the microchannel has a characteristic distance between 500 μm and 1500 μm. In one embodiment, the microchannel has a characteristic distance of between 800 and 1200 μm. In one embodiment, the microchannel has a characteristic distance between 100 μm and 500 μm. In one embodiment, the microchannel has a characteristic distance of between 100 and 300 μm. The microfluidic channel can also be a cylindrical channel, the diameter of which is the characteristic distance.
“Microfluidic”: relates to a structure comprising at least one microchannel In one embodiment, microfluidic relates to a structure comprising at least two microchannels. In one embodiment, microfluidic relates to a structure comprising at least three microchannels.
“Lipid nanocapsules”: concerns a nanoparticle consisting of a liquid or semi-liquid core at room temperature, coated with a film that is solid at room temperature. For the purposes of the invention, the lipid nanocapsules comprise a core consisting of one or more fatty substances and a crown consisting of one or more surfactants and/or cosurfactants. In one embodiment, the lipid nanocapsules have a particle size between 15 and 120 nm. In one embodiment, the lipid nanocapsules have a particle size between 15 and 70 nm. In one embodiment, the lipid nanocapsules have a particle size between 20 and 120 nm. In one embodiment, the lipid nanocapsules have a particle size between 20 and 100 nm. In one embodiment, the lipid nanocapsules have a particle size between 20 and 50 nm. In one embodiment, the lipid nanocapsules have a particle size between 50 and 100 nm. In one embodiment, the lipid nanocapsules have a particle size between 15 and 50 nm. In one embodiment, the lipid nanocapsules have a particle size between 20 and 35 nm. In one embodiment, the lipid nanocapsules have a particle size between 35 and 50 nm.
“Nano-emulsification”: refers to a process consisting in dispersing two immiscible liquid phases, such as water and oil, but which through specific operations succeed in having a macroscopically homogeneous, but microscopically heterogeneous, appearance. One of the phases will be dispersed in the second phase in the form of nano-droplets or liquid nano-particles. For the purposes of the invention, the lipid nano-droplets or nano-particles are lipid nanocapsules as described above.
“Nano-emulsion”: relates to an emulsion produced by nano-emulsification, composed of nano-droplets or nano-particles with a size ranging from 15 nm to 120 nm. In one embodiment, a nano-emulsion is an emulsion that includes nano-droplets or nano-particles with a size in the range of 15 nm to 70 nm. In one embodiment, a nano-emulsion is an emulsion that includes nano-droplets or nano-particles with a size in the range of 20 nm to 120 nm. In one embodiment, a nano-emulsion is an emulsion that includes nano-droplets or nano-particles with a size in the range of 20 nm to 100 nm. In one embodiment, a nano-emulsion is an emulsion that includes nano-droplets or nano-particles with a size in the range of 20 nm to 50 nm. For the purposes of the invention, the lipid nano-droplets or nano-particles are lipid nanocapsules as described above
“Fatty phase” or “oily phase” are equivalent terms. They denote a phase comprising at least 50% of one or more fatty substances immiscible in water at 25° C. and atmospheric pressure. In one embodiment, they denote a phase comprising at least 60% of one or more fatty substances immiscible in water at 25° C. and atmospheric pressure. In one embodiment, they denote a phase comprising at least 70% of one or more fatty substances immiscible in water at 25° C. and atmospheric pressure. In one embodiment, they denote a phase comprising at least 80% of one or more fatty substances immiscible in water at 25° C. and atmospheric pressure.
“Pharmacologically active molecule”: relates to a compound of therapeutic interest. Primarily, a pharmacologically active molecule may be indicated for the treatment or prevention of diseases.
“Treatment of a disease” refers to the reduction or elimination of at least one side effect or symptom of a disease, disorder or condition associated with the impairment of an organ, tissue or cellular function. The term “preventing a disease” refers to preventing the onset of a symptom.
“Heat-sensitive active agent” relates, within the meaning of the invention, to a molecule that can undergo a change in its chemical structure due to the rise in temperature. In one embodiment, the molecule is cut into one or more fragments. In one embodiment, the molecule undergoes degradation of its biological activity. In one embodiment, the molecule undergoes degradation of its pharmacological activity. For the purposes of the invention, the heat-sensitive active agent exhibits a sensitivity to a temperature above 70° C., preferably above 50° C. In one embodiment, the heat-sensitive active agent is a pharmacologically active ingredient. In one embodiment, the heat-sensitive active agent is a heat-sensitive cosmetic active. In one embodiment, the heat-sensitive active agent is selected from peptides, proteins, nucleic acids, anticancer agents, anti-infective agents or antibiotics.
“Surfactant”: concerns an amphiphilic compound which, by virtue of this particular structure, makes it possible to lower the free energy of interfaces, for example oil/water or air/water interfaces. Thus a surfactant is a compound which modifies the interfacial tension between two surfaces. Surfactants facilitate the formation of drops or bubbles by reducing the interfacial tension.
“SOR”: designates the weight ratio of the flow rate of surfactants and co-surfactants to the flow rate of fatty substances in the formulation chamber.
“SOWR”: designates the weight ratio of the sum of the flow rates of surfactants, co-surfactants and fatty substances to the flow rate of the aqueous phase in the formulation chamber.

### DETAILED DESCRIPTION

The present invention relates to a continuous nano-emulsification process characterized in that said process is carried out by concentration phase inversion (CPI) in a microfluidic reactor, and comprising the following steps:
a. injection into a first microchannel of an aqueous phase, said first microchannel opening into a formulation chamber,
b. injection into a second microchannel of a fatty phase comprising one or more fatty substances, and one or more surfactants, said second microchannel opening into said formulation chamber, then
c. mixing of the aqueous phase and the fatty phase in said formulation chamber, then
d. recovery, at the outlet of the formulation chamber, of a suspension comprising lipid nanocapsules.

The process according to the invention comprises a step of injecting an aqueous phase into a first microchannel, the first microchannel opening into a formulation chamber.

In one embodiment, the aqueous phase comprises at least 90% by weight of water. In one embodiment, the aqueous phase comprises at least 95% by weight of water. In one embodiment, the aqueous phase comprises at least 98% by weight of water. In one embodiment, the aqueous phase consists of water. In one embodiment, the water is MilliQ ultrapure water filtered through 0.2 μm.

In one embodiment, the aqueous phase further comprises an active agent. In a preferred embodiment, the active agent is a heat-sensitive active. In a preferred embodiment, the active agent is a heat-sensitive pharmacologically active ingredient. In one embodiment, the active agent is a heat-sensitive cosmetic active. In a preferred embodiment, the heat-sensitive active agent is hydrophilic in nature. In one embodiment, the heat-sensitive active agent is selected from peptides, proteins, nucleic acids, anticancer agents or anti-infective agents.

In one embodiment, the aqueous phase is injected into the formulation chamber.

In one embodiment, the aqueous phase is prepared in a mixing chamber. The outlet of the mixing chamber is connected via said first microchannel to the formulation chamber.

In one embodiment, the mixing chamber is of the static mixer type, that is to say a device for continuously mixing aqueous phases. The outlet of the mixing chamber is connected via said first microchannel to the formulation chamber.

In one embodiment, the mixing chamber is of the stirred tank type, that is, the aqueous phases are mixed by mechanical action. The outlet of the mixing chamber is connected via said first microchannel to the formulation chamber.

In one embodiment, the first microchannel consists of a polymer. In one embodiment, the first microchannel consists of a polymer selected from polyaryletherketones (PEAK). In one embodiment, the first microchannel consists of polyetheretherketone (PEEK).

In one embodiment, the first microchannel consists of silica.

In one embodiment, the first microchannel consists of silicon.

In one embodiment, the first microchannel consists of glass.

In one embodiment, the first microchannel consists of polytetrafluoroethylene (PTFE).

In one embodiment, the first microchannel is a parallelepipedal channel. When the first microchannel is a parallelepipedal channel, the characteristic distances of the channel are depth and width. In one embodiment, the first microchannel has a depth of between 100 μm and 1500 μm. In one embodiment, the first microchannel has a width of between 100 μm and 1500 μm.

In one embodiment, the first microchannel is a cylindrical channel. When the first microchannel is a cylindrical channel, the characteristic distance of the channel is the diameter. In one embodiment, the first microchannel has a characteristic distance between 200 μm and 2000 μm. In one embodiment, the first microchannel has a characteristic distance between 500 μm and 1500 μm. In one embodiment, the first microchannel has a characteristic distance between 800 and 1200 μm.

In one embodiment, the injection of the aqueous phase into the first microchannel is by means of a syringe pump. In one embodiment, the injection of the aqueous phase into the first microchannel is by means of a ISCO 100DX syringe pump. In one embodiment, the injection of the aqueous phase into the first microchannel is by means of a Harvard Apparatus PHD Ultra syringe pump. In one embodiment, the injection of the aqueous phase into the first microchannel is by means of an Elveflow OBI MK3 pressure controller.

In one embodiment, the flow rate of aqueous phase in the first microchannel is between 100 μL/min and 500,000 μL/min, preferably between 1000 μL/min and 72,500 μL/min.

In one embodiment, the first microchannel is thermalized, that is to say permanently maintained at a set temperature. In one embodiment, the first microchannel is thermalized by a water circulation system via the use of a thermostatic bath.

In one embodiment, the first microchannel is thermalized at a temperature between 20° C. and 70° C. In one embodiment, the first microchannel is thermalized at a temperature of 20° C., 25° C., 30° C., 35° C., 40° C., 45° C., 50° C., 55° C., 60° C., 65° C., 70° C. the first microchannel is thermalized at a temperature between 20° C. and 30° C. In a preferred embodiment, the first microchannel is thermalized at a temperature between 30° C. and 50° C. The process according to the invention comprises a step of injection into a second microchannel of a fatty phase comprising one or more fatty substances immiscible in said aqueous phase, and one or more surfactants, the second microchannel opening into the formulation chamber.

In one embodiment, the fatty phase is injected into the formulation chamber.

In one embodiment, the fatty phase is prepared in a mixing chamber. The outlet of the mixing chamber is connected via said second microchannel to the formulation chamber.

In one embodiment, the mixing chamber is of the static mixer type, that is to say a device for continuously mixing fatty phases. The output of the static mixer is connected via said second microchannel to the formulation chamber.

In one embodiment, the mixing chamber is of the stirred tank type, that is to say that the fatty phases are mixed by mechanical action. The outlet of the stirred tank is connected via said second microchannel to the formulation chamber.

In one embodiment, the weight ratio of the sum of the flow rates of surfactants and co-surfactants to the flow rate of fatty substances (S OR) in the formulation chamber is between 0.8 and 4. In one embodiment, the weight ratio of the sum of the flow rates of surfactants and co-surfactants to the flow rate of fatty substances (SOR) in the formulation chamber is between 2 and 4.

In one embodiment, the second microchannel consists of a polymer. In one embodiment, the second microchannel consists of a polymer selected from polyaryletherketones (PEAK). In one embodiment, the second microchannel is made of polyetheretherketone (PEEK).

In one embodiment, the second microchannel consists of silica.

In one embodiment, the second microchannel consists of silicon.

In one embodiment, the second microchannel consists of glass.

In one embodiment, the second microchannel is a parallelepipedal channel. When the second microchannel is a parallelepipedal canal, the characteristic distances of the canal are depth and width. In one embodiment, the second microchannel has a depth between 100 μm and 1500 μm. In one embodiment, the second microchannel has a width of between 100 μm and 1500 μm.

In one embodiment, the second microchannel is a cylindrical channel. When the second microchannel is a cylindrical channel, the characteristic distance of the channel is the diameter. In one embodiment, the second microchannel has a characteristic distance between 100 μm and 1500 μm. In one embodiment, the second microchannel has a characteristic distance between 500 μm and 1500 μm. In one embodiment, the second microchannel has a characteristic distance between 800 and 1200 μm. In one embodiment, the second microchannel has a characteristic distance between 100 μm and 500 μm. In a mode of embodiment, the second microchannel has a characteristic distance of between 100 and 300 μm.

In one embodiment, the injection of the fatty phase into the second microchannel is carried out by means of a syringe pump. In one embodiment, the injection of the fatty phase into the second microchannel is performed by means of an ISCO 100DX syringe pump. In one embodiment, the injection of the fatty phase into the second microchannel is by means of a Harvard Apparatus PHD 2000 infusion syringe pump. In one embodiment, the injection of the aqueous phase into the second microchannel is by means of an Elveflow OBI MK3 pressure controller.

In one embodiment, the flow rate of fatty phase in the second microchannel is between 50 μL/min and 500,000 μL/min. In one embodiment, the flow rate of fatty phase in the second microchannel is between 300 μL/min and 10,000 μL/min In one embodiment, the flow rate of fatty phase in the second microchannel is between 100 μL/min and 500,000 μL/min. In one embodiment, the flow rate of fatty phase in the second microchannel is between 50 μL/min and 300 μL/min. In one embodiment, the flow rate of fatty phase in the second microchannel is between 100 μL/min and 300 μL/min In one embodiment, the flow rate of fatty phase in the second microchannel is between 300 μL/min and 500 μL/min. In one embodiment, the flow rate of fatty phase in the second microchannel is between 500 μL/min and 1000 μL/min.

In one embodiment, the second microchannel is thermalized, that is to say permanently maintained at a set temperature. In one embodiment, the second microchannel is thermalized by a water circulation system via the use of a thermostatic bath.

In one embodiment, the second microchannel is thermalized at a temperature between 20° C. and 70° C. In one embodiment, the second microchannel is thermalized at a temperature of 20° C., 25° C., 30° C., 35° C., 40° C., 45° C., 50° C., 55° C., 60° C., 65° C., 70 ° C. the second microchannel is thermalized at a temperature between 20° C. and 30° C. In a preferred embodiment, the second microchannel is thermalized at a temperature between 30° C. and 50° C. In one embodiment, the surfactant(s) of the fatty phase are chosen from nonionic hydrophilic surfactants, and mixtures thereof. In one embodiment, the surfactant(s) of the fatty phase are chosen from mono- and di-esters of fatty acid and of polyethylene glycol, and mixtures thereof. In one embodiment, the surfactant(s) of the fatty phase are chosen from mono- and di-esters of stearic acid and of polyethylene glycol, and mixtures thereof. In one embodiment, the fatty phase surfactant is Kolliphor® HS 15 from BASF.

In one embodiment, the fatty phase comprises one or more surfactants in a content of between 40% and 65% by weight, relative to the total weight of the fatty phase. In one embodiment, the fatty phase comprises one or more surfactants in a content of between 45% and 65% by weight, relative to the total weight of the fatty phase. In one embodiment, the fatty phase comprises one or more surfactants in a content of between 45% and 55% by weight, relative to the total weight of the fatty phase. In one embodiment, the fatty phase comprises one or more surfactants in a content of between 55% and 65% by weight, relative to the total weight of the fatty phase.

In one embodiment, the fatty substance(s) of the fatty phase are chosen from glycerol mono-esters, di-esters and tri-esters, polyethylene glycol mono-esters and di-esters, and mixtures thereof. In one embodiment, the fatty substance(s) of the fatty phase are chosen from C₈-C₁₈ triglycerides, and mixtures thereof. In one embodiment, the fatty substance (s) are chosen from triglycerides of capric and caprylic acids and their mixtures. In one embodiment, the fatty substance of the fatty phase is Labrafac® WL 1349 from Gattefosse. In one embodiment, the fatty substance of the fatty phase is Captex® 8000 from Abitec. In one embodiment, the fatty substance of the fatty phase is Labrafil® M1944 CS from Gattefosse (mixture of mono-, di- and triglycerides, PEG-6, oleate of mono- and di-triesters). In one embodiment, the fatty substance of the fatty phase is Ethyl Oleate. In one embodiment, the fatty substance of the fatty phase is Ethyl Palmitate. In one embodiment, the fatty substance of the fatty phase is Glyceryl Oleate.

In one embodiment, the fatty phase comprises one or more fatty substances in a content of between 20% and 60% by weight, relative to the total weight of the fatty phase. In one embodiment, the fatty phase comprises one or more fatty substances in a content of between 25% and 55% by weight, relative to the total weight of the fatty phase. In one embodiment, the fatty phase comprises one or more fatty substances in a content of between 25% and 35% by weight, relative to the total weight of the fatty phase. In one embodiment, the fatty phase comprises one or more fatty substances in a content of between 35% and 55% by weight, relative to the total weight of the fatty phase.

In one embodiment, the fatty phase further comprises one or more co-surfactants. In one embodiment, the co-surfactant(s) are chosen from lipophilic surfactants and their mixtures. In one embodiment, the co-surfactant of the fatty phase is a phospholipid selected from lecithins, phosphatilglycerol, phophatidylinositol, phosphatidylserine, phophatidic acid, phosphatidylethanolamine and their mixtures. In a preferred embodiment, the co-surfactant(s) are chosen from nonionic surfactants and mixtures thereof. In one embodiment, the co-surfactant is chosen from sorbitan esters. In one embodiment, the co-surfactant is sorbitan monooleate. In one embodiment, the co-surfactant is Span® 80 from BASF. In one embodiment, the co-surfactant is diethylene glycol mono-ethyl ether. In one embodiment, the co-surfactant is Transcutol® HP from Gattefossé.

In one embodiment, the fatty phase further comprises one or more co-surfactants in a content of between 0% and 20% by weight, relative to the total weight of the fatty phase. In one embodiment, the fatty phase further comprises one or more co-surfactants in a content of between 0% and 10% by weight, relative to the total weight of the fatty phase. In one embodiment, the fatty phase further comprises one or more co-surfactants in a content of between 10% and 20% by weight, relative to the total weight of the fatty phase.

In one embodiment, the fatty phase further comprises water. In one embodiment, the fatty phase further comprises water in a content of between 0% and 30% by weight, relative to the total weight of the fatty phase. In one embodiment, the fatty phase further comprises water in a content of between 0% to 20%. In one embodiment, the fatty phase further comprises water in a content of between 0% to 15%.

In one embodiment, the fatty phase further comprises an active agent. In a preferred embodiment, the active agent is a heat-sensitive active. In a preferred embodiment, the active agent is a heat-sensitive pharmacologically active ingredient. In one embodiment, the active agent is a heat-sensitive cosmetic active. In a preferred embodiment, the heat-sensitive active agent is hydrophilic in nature. In another preferred embodiment, the heat-sensitive active agent is lipophilic in nature. In one embodiment, the heat-sensitive active agent is chosen from among peptides, proteins or nucleic acids, anticancer agents or anti-infective agents.

The process according to the invention comprises a step c of mixing the aqueous phase and the fatty phase in the formulation chamber.

In one embodiment, the formulation chamber is of the “co-flow” type, that is to say that the flow of the first microchannel and the flow of the second microchannel are in the same direction and open into the formulation chamber from the same direction. In one embodiment, the formulation chamber is of the “co-flow” type and the first microchannel has a larger diameter than that of the second microchannel. In one embodiment, the formulation chamber is of the “co-flow” type and the first microchannel includes the second microchannel.

In one embodiment, the formulation chamber is of “T” type, that is to say that the flow of the first microchannel and the flow of the second microchannel in the formulation chamber form a “T” with the flow of output channel In one embodiment, the flow of the first microchannel and the flow of the second microchannel in the mixing chamber form an angle of between 30° and 150° with the flow of the outlet channel. In one embodiment, the flow of the first microchannel and the flow of the second microchannel in the mixing chamber form an angle comprised of 45° with the flow of the outlet channel.

In one embodiment, the flow of the first microchannel and the flow of the second microchannel in the mixing chamber form an angle of 135° with the flow of the outlet channel.

In one embodiment, the formulation chamber is of the “Flow focusing” type (also called hydrodynamic focusing), that is to say that the flow of the first microchannel is focused in a constriction by the flow of a second microchannel and of a third microchannel. In one embodiment, the flow of the first microchannel and the flow of the second or third microchannel in the mixing chamber form an angle of between 15° and 90°.

In one embodiment, the first microchannel is thermalized at a temperature between 20° C. and 70° C. In one embodiment, the first microchannel is thermalized at a temperature of 20° C., 25° C., 30° C., 35° C., 40° C., 45° C., 50° C., 55° C., 60° C., 65° C., 70° C. the first microchannel is thermalized at a temperature between 20° C. and 30° C. In a preferred embodiment, the first microchannel is thermalized at a temperature between 30° C. and 50° C. In one embodiment, the weight ratio of the sum of the flow rates of surfactants, co-surfactants and fatty substances to the flow rate of the aqueous phase (SOWR) in the formulation chamber is between 0.01 and 0.30. In one embodiment, the weight ratio of the sum of the flow rates of surfactant, co-surfactants and fatty substances to the flow rate of the aqueous phase (SOWR) in the formulation chamber is between 0.03 and 0.3, preferably is between 0.04 and 0.2.

The process according to the invention comprises a step of recovering, at the outlet from the formulation chamber, a suspension comprising lipid nanocapsules in an aqueous phase.

Advantageously, the process of the present invention as described above allows the formulation of lipid nanocapsule at low temperature. This embodiment is particularly advantageous for the encapsulation of heat-sensitive pharmacologically active ingredients. The lipid nanocapsules obtained at low temperature also exhibit a lower polydispersity, allowing the production of nanomedicines of uniform size for optimal cell targeting and internalization efficiency.

In one embodiment, the lipid nanocapsules have a particle size between 15 and 120 nm.

In one embodiment, the lipid nanocapsules have a particle size between 15 and 70 nm. In one embodiment, the lipid nanocapsules have a particle size between 20 and 120 nm. In one embodiment, the lipid nanocapsules have a particle size between 20 and 100 nm. In one embodiment, the lipid nanocapsules have a particle size between 15 and 50 nm. In one embodiment, the lipid nanocapsules have a particle size between 20 and 50 nm. In one embodiment, the lipid nanocapsules have a particle size between 50 and 100 nm. In one embodiment, the lipid nanocapsules have a particle size between 20 and 35 nm. In one embodiment, the lipid nanocapsules have a particle size between 35 and 50 nm.

In one embodiment, the lipid nanocapsules have a polydispersity index of between 0.05 and 0.2. In one embodiment, the lipid nanocapsules have a polydispersity index of between 0.05 and 0.15. In one embodiment, the lipid nanocapsules have a polydispersity index of between 0.05 and 0.1.

The invention also relates to lipid nanocapsules obtainable by the process according to the invention.

In one embodiment, the lipid nanocapsules comprise a core consisting of one or more fatty substances and a crown consisting of one or more surfactants and/or co-surfactants. The lipid nanocapsules are metastable and withstand a dilution for which the concentration of the surfactants is lower than their critical micellar concentration.

In one embodiment, the fatty substance(s) are chosen from glycerol mono-esters, di-esters and tri-esters, polyethylene glycol mono-esters and di-esters, and mixtures thereof, preferably from C₈-C₁₈ triglycerides, and mixtures thereof, more preferably from the triglycerides of capric and caprylic acids and their mixtures.

In one embodiment, the surfactant (s) are chosen from nonionic hydrophilic surfactants, and mixtures thereof, preferably from mono- and di-esters of fatty acid and of polyethylene glycol, and mixtures thereof, more preferably from mono- and di-esters of stearic acid and of polyethylene glycol, and mixtures thereof.

In one embodiment, the co-surfactant (s) are chosen from nonionic surfactants, preferably from sorbitan monooleate or diethylene glycol mono-ethyl ether, and mixtures thereof.

In one embodiment, the lipid nanocapsules of the invention further include an active agent. In a preferred embodiment, the active agent is a heat-sensitive active. In a preferred embodiment, the active agent is a heat-sensitive pharmacologically active ingredient. In one embodiment, the active agent is a heat-sensitive cosmetic active. In one embodiment, the heat-sensitive active agent is selected from peptides, proteins, nucleic acids, anticancer agents or anti-infective agents.

In one embodiment, the lipid nanocapsules of the invention are part of the composition of a medicament for administration.

In one embodiment, the lipid nanocapsules of the invention form part of the composition of a medicament intended to be administered enterally, for example orally, rectally or buccally.

In one embodiment, the lipid nanocapsules of the invention form part of the composition of a medicament intended to be administered percutaneously, for example by transdermal or cutaneous route.

In one embodiment, the lipid nanocapsules of the invention form part of the composition of a medicament intended to be administered by air, for example by nasal, auricular or pulmonary route.

In one embodiment, the lipid nanocapsules of the invention form part of the composition of a medicament intended to be administered by the ocular route.

In one embodiment, the lipid nanocapsules of the invention form part of the composition of a medicament intended to be administered by the vaginal route.

In one embodiment, the lipid nanocapsules of the invention form part of the composition of a medicament intended to be administered parenterally, for example intravenously, intraarterially, intradermally, epidural, subcutaneously.

In one embodiment, the lipid nanocapsules of the invention enter into the composition of a cosmetic product intended to be administered.

In one embodiment, the lipid nanocapsules have a particle size between 15 and 120 nm. In one embodiment, the lipid nanocapsules have a particle size between 15 and 70 nm. In one embodiment, the lipid nanocapsules have a particle size between 20 and 120 nm. In one embodiment, the lipid nanocapsules have a particle size between 20 and 100 nm. In one embodiment, the lipid nanocapsules have a particle size between 15 and 50 nm. In one embodiment, the lipid nanocapsules have a particle size between 20 and 50 nm. In one embodiment, the lipid nanocapsules have a particle size between 50 and 100 nm. In one embodiment, the lipid nanocapsules have a particle size between 20 and 35 nm. In one embodiment, the lipid nanocapsules have a particle size between 35 and 50 nm.

In one embodiment, the lipid nanocapsules have a polydispersity index of between 0.05 and 0.2. In one embodiment, the lipid nanocapsules have a polydispersity index of between 0.05 and 0.15. In one embodiment, the lipid nanocapsules have a polydispersity index of between 0.05 and 0.1.

The sizes of lipid nanocapsules are measured by the dynamic light scattering method (DLS method).

The invention also relates to the use of the lipid nanocapsules according to the invention as active agent nanovectors.

In one embodiment, the active agent is a pharmacologically active ingredient. In one embodiment, the active agent is a cosmetic active.

The invention also relates to the use of the lipid nanocapsules according to the invention as nanovectors of pharmacologically active ingredient.

In one embodiment, the pharmacologically active ingredient is a heat-sensitive active.

In one embodiment, the pharmacologically active ingredient is chosen from proteins, peptides, oligonucleotides and DNA plasmids.

In one embodiment, the pharmacologically active ingredient is chosen from anti-infectives, for example antimycotics and antibiotics.

In one embodiment, the pharmacologically active ingredient is chosen from anticancer drugs.

In one embodiment, the pharmacologically active ingredient is chosen from active ingredients intended for the Central Nervous System, such as antiparkinson drugs and more generally active ingredients for treating neurodegenerative diseases.

In one embodiment, the pharmacologically active ingredient is lipophilic in nature.

In one embodiment, the pharmacologically active ingredient is dissolved or dispersed in the core of the lipid nanocapsules.

In one embodiment, the pharmacologically active ingredient is incorporated into the core of the nanocapsule. In one embodiment, the pharmacologically active ingredient is incorporated into the fatty phase.

In one embodiment, the pharmacologically active ingredient is fixed to the surface of the lipid nanocapsules.

In one embodiment, the pharmacologically active ingredient is water-soluble or dispersible in the aqueous phase.

In one embodiment, the water-soluble or dispersible in the aqueous phase pharmacologically active ingredient is fixed to the surface of the lipid nanocapsules by introducing said active ingredient into the solution in which the stable lipid nanoparticles obtained at the outcome of the process according to the invention are dispersed. In one embodiment, the water-soluble or dispersible in the aqueous phase pharmacologically active ingredient is fixed to the surface of the lipid nanocapsules by introducing said pharmacologically active ingredient into the water included in the fatty phase before the formulation of the stable lipid nanoparticles obtained at the outcome of the process according to the invention.

The invention also relates to the use of the lipid nanocapsules according to the invention as cosmetic active nanovectors.

In one embodiment, the cosmetic active is a heat-sensitive active.

The invention also relates to lipid nanocapsules according to the invention for their use as a medicament.

The invention also relates to the use of the lipid nanocapsules according to the invention in the manufacture of a medicament.

The invention also relates to a method of treating a subject in need thereof, said method comprising administering to said subject a therapeutically effective amount of at least one lipid nanocapsule according to the invention.

### BRIEF DESCRIPTION OF THE DRAWINGS

FIG. 1 is a diagram of the device used in the process according to the invention according to a first embodiment (called “co-flow” type). Microchannel 1 allows the aqueous phase to be injected into formulation chamber 3. Microchannel 2 allows the fatty phase to be injected into formulation chamber 3. The formulation chamber 3 thus makes it possible to form the lipid nanocapsules according to the invention.

FIG. 2 compares the results in terms of size of lipid nanoparticles and polydispersity index of a microfluidic process according to the present invention and a comparative batch process.

FIG. 3 compares the results in terms of size of lipid nanoparticles and polydispersity index of a microfluidic process according to the present invention at different flow rates.

FIG. 4 is a diagram of the device used in the process according to the invention according to a second embodiment (called “T at 45°” type). The microchannel 4 and the microchannel 5 of the mixing chamber form an angle of 45° with the microchannel 7. Microchannel 4 allows the aqueous phase to be injected into formulation chamber 6. Microchannel 5 allows the fatty phase to be injected into formulation chamber 6. The formulation chamber 6 thus makes it possible to form the lipid nanocapsules according to the invention and to recover them via the microchannel 7.

FIG. 5 is a diagram of the device used in the process according to the invention according to a third embodiment (called “T” type). The microchannel 8 and the microchannel 9 of the mixing chamber form an angle of 135° with the microchannel 10 of the outlet. The microchannel 8 allows the aqueous phase to be injected into the formulation chamber 12. The microchannel 9 makes it possible to inject the fatty phase into the formulation chamber 12. The baffles 11 serve to create additional mixing zones in the microchannel 10. The formulation chamber 12 thus makes it possible to form the lipid nanocapsules according to the invention and to recover them via the outlet microchannel 10.

FIG. 6 is a diagram of the device used in the process according to the invention according to a second embodiment (referred to as the “Flow focusing” type formulation chamber). The microchannel 14 is focused in a constriction by the flow of the microchannel 13 and of the microchannel 15 of the mixing chamber 17. The microchannels 13 and 15 form an angle of 90° with the microchannel 14. The microchannel 14 makes it possible to inject the fatty phase into the formulation chamber 17. The microchannels 13 and 15 make it possible to inject the aqueous phase into the formulation chamber 17 with the same flow rate in each of the microchannels. The formulation chamber 17 thus makes it possible to form the lipid nanocapsules according to the invention and to recover them via the microchannel 16.

### EXAMPLES

The present invention will be better understood on reading the following examples which illustrate the invention without limitation.

### Example 1

### Formulation of Lipid Nanocapsules by Continuous CPI Process Using a “Co-Flow” Type Device

### Reagents and Products Used

Kolliphor® HS 15: PEG 660 12-hydroxystearate sold by BASF,
Labrafac WL 1349: Triglycerides of capric and caprylic acids sold by Gattefosse,
MilliQ ultrapure water: prepared using a Millipore device,
Tetrahydrofuran: used for cleaning the microfluidic system.

### Specific Material and Conditions

Weighing scale: for the preparation of the formulation,
Heating/stirring plate IKA C-MAG HS7: for the preparation of the formulation,
Fisherband Polystat 36 thermostatic bath, to maintain the oily phase at temperature and to thermostate the injection capillaries,
Silica capillary with an internal diameter of 320 μm, for supplying the formulation chamber with the fatty phase,
Silica capillary with an internal diameter of 530 μm, for supplying the formulation chamber with the aqueous phase,
Silica capillary with an internal diameter of 530 μm, for the exit of the mixture from the mixture formulation,
Harvard Apparatus PHD 2000 infusion syringe pump to inject the aqueous phase into the formulation chamber,
ISCO 100 DX syringe pump for injecting the fatty phase into the formulation chamber.

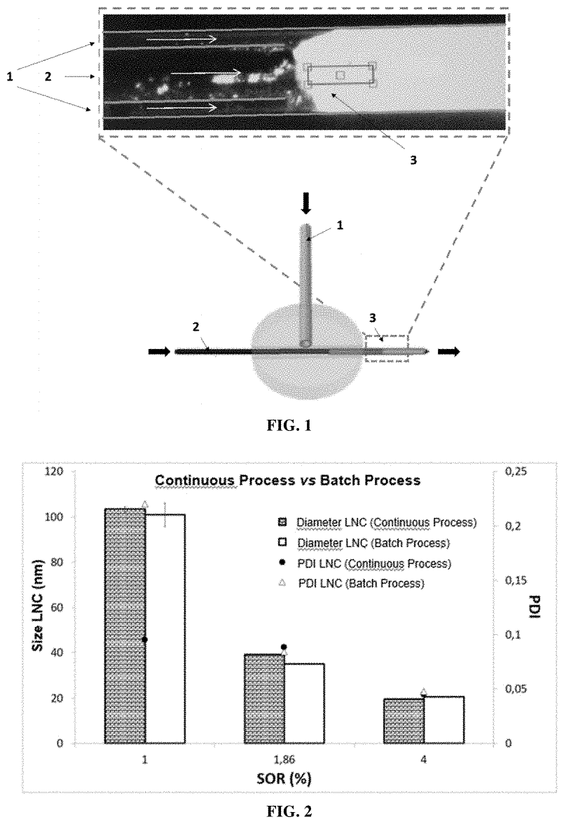

The device used in this first example is shown in FIG. 1.

The first microchannel 1 (internal diameter 530 μm) allows the injection of the aqueous phase consisting of MilliQ ultrapure water. The second microchannel 2 (internal diameter 320 μm) allows the injection of the fatty phase consisting of a fatty substance, Labrafac® WL 1349, and a surfactant, Kolliphor® HS 15. Both microchannels 1 and 2 are connected to a T junction and are arranged in the same plane at 90° to each other. A microchannel 3 (internal diameter 530 μm) from the mixer outlet is connected to the junction fitting T so that the microchannel 2 is introduced into the capillary 3, leading to the mixing zone of the fatty and aqueous phases where the formation of lipid nanocapsules takes place. The flow rates for both microchannels 1 and 2 are set using the ISCO 100 DX Syringe Pump and Harvard Apparatus PHD 2000 Infusion Syringe Pump, respectively.

On the basis of this device, the characteristics of the lipid nanocapsules, size and polydispersity index, of 3 formulations (by concentration phase inversion) of lipid nanocapsules obtained by a comparative batch process and a continuous process according to the invention were compared.

### Formulations

TABLE 1

| | (% by weight) | | |
|---|---|---|---|
| | Kolliphor ® | Labrafac ®WL | ultrapure |
| SOR | HS15 | 1349 | water |
| 1 | 2.245 | 2.245 | 95.51 |
| 1.86 | 2.918 | 1.572 | 95.51 |
| 4 | 3.592 | 0.898 | 95.51 |

FIG. 2 presents the results of the characteristics of the lipid nanocapsules, size and polydispersity index.

It is found that the size of the particles is substantially equivalent for the lipid nanocapsules produced by the comparative batch process as for the lipid nanocapsules produced by the continuous process according to the invention.

However, the polydispersity index is significantly reduced for the continuous process according to the invention, in particular for an SOR ratio of 1.

The process according to the invention therefore makes it possible to obtain lipid nanocapsules which are of controlled sizes and relatively very monodisperse, which is particularly suitable for the vectorization of pharmaceutical compounds. In addition, the process according to the invention makes it possible to be industrialized more easily by placing continuous reactors in parallel.

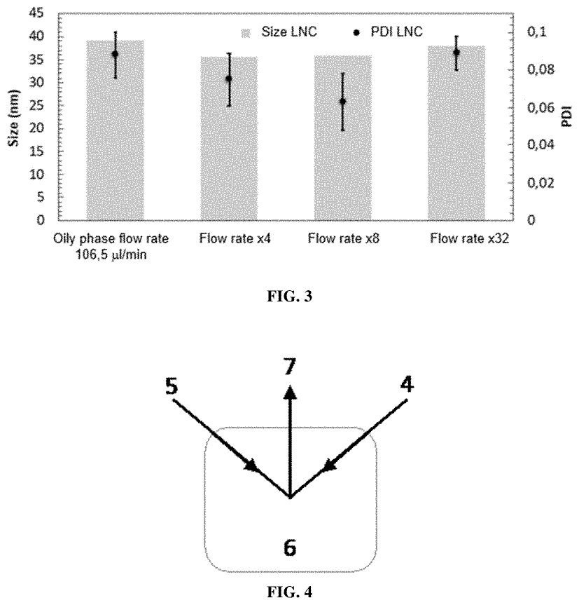

FIG. 3 shows the effect of flow rate on the size and polydispersity index of lipid nanocapsules. This figure shows that increasing the flow rate makes it possible to slightly reduce the size of the particles but to significantly reduce the polydispersity index.

### Example 2

### Formulation of Lipid Nanocapsules by Continuous CPI Process Using a “T” Type Formulation Chamber and Syringe Pumps

### Reagents and Products Used

Kolliphor® HS 15: PEG 660 12-hydroxystearate sold by BASF,
Labrafac®WL 1349: Triglycerides of capric and caprylic acids sold by Gattefosse,
Span® 80: Sorbitan monooleate sold by BASF,
MilliQ ultrapure water: prepared using a WATERS device,
Tetrahydrofuran: used for cleaning the microfluidic system.

### Specific Material and Conditions

Mettler Toledo weighing scale: for the preparation of the formulation,
Heating/stirring plate IKA C-MAG HS7: for the preparation of the formulation,
Polyetheretherketone (PEEK) capillary with an internal diameter of 1 mm, for supplying the formulation chamber with the fatty phase,
Polyetheretherketone (PEEK) capillary with an internal diameter of 1 mm, for supplying the formulation chamber with the aqueous phase,
Polyetheretherketone (PEEK) capillary with an internal diameter of 1 mm, for the exit of the mixture from the formulation chamber,
Harvard Apparatus PHD 2000 infusion syringe pump to inject the fatty phase into the formulation chamber,
Harvard Apparatus PHD Ultra (or ISCO 100DX) syringe pump for injecting the aqueous phase into the formulation chamber.

The device used in this second example is shown in FIG. 4.

The first microchannel 4 allows the injection of the aqueous phase consisting of MilliQ ultrapure water filtered at 0.2 μm. The second microchannel 5 allows the injection of the fatty phase consisting of a fatty substance, Labrafac WL 1349, a surfactant, Kolliphor® HS 15 and optionally a co-surfactant, Span® 80. Both microchannels 4 and 5 are arranged in the same plane at 45° to each other and are each connected at one of their ends to a syringe pump allowing the flow control of each of the phases. Both microchannels 4 and 5 open at their other ends into a formulation chamber 6 where the formation of the lipid nanocapsules takes place. The suspension comprising the lipid nanocapsules is recovered through microchannel 7.

### Batch/Continuous Comparison

Table 2 below shows comparative tests of the results of the mixing plan of different fatty phase formulations obtained by batch and continuous process.

The temperature was set at 50° C., the oil phase flow rate at 425 μL/min and the SOWR ratio at 0.047.

TABLE 2

| Fatty phase composition (% by weight) | | | Continuous CPI | | Batch CPI | |
|---|---|---|---|---|---|---|
| Kolliphor ® | Labrafac ®WL | Span ® | Size | | Size | |
| HS15 | 1349 | 80 | (nm) | PDI | (nm) | PDI |
| 45 | 55 | 0 | 96.2 | 0.08 | 107.5 | 0.14 |
| 55 | 45 | 0 | 59.5 | 0.11 | 66.6 | 0.17 |
| 65 | 35 | 0 | 34.9 | 0.13 | 38.1 | 0.20 |
| 45 | 50 | 5 | 66.4 | 0.09 | 71.0 | 0.15 |
| 50 | 45 | 5 | 56.3 | 0.09 | 64.6 | 0.19 |
| 55 | 40 | 5 | 43.4 | 0.07 | 47.0 | 0.12 |
| 57.5 | 37.5 | 5 | 39.5 | 0.07 | 41.4 | 0.15 |
| 65 | 30 | 5 | 29.4 | 0.06 | 39.0 | 0.25 |
| 50 | 40 | 10 | 44.5 | 0.07 | 44.3 | 0.10 |
| 57.5 | 32.5 | 10 | 31.8 | 0.06 | 35.1 | 0.17 |
| 60 | 30 | 10 | 30.1 | 0.06 | 30.6 | 0.11 |
| 65 | 25 | 10 | 26.5 | 0.07 | 28.0 | 0.18 |
| 45 | 40 | 15 | 61.0 | 0.18 | 55.7 | 0.12 |
| 50 | 35 | 15 | 37.3 | 0.06 | 39.8 | 0.14 |
| 52.5 | 32.5 | 15 | 32.7 | 0.05 | 39.3 | 0.22 |
| 60 | 25 | 15 | 27.1 | 0.05 | 41.7 | 0.30 |
| 45 | 35 | 20 | 39.8 | 0.12 | 42.2 | 0.15 |
| 55 | 25 | 20 | 28.3 | 0.14 | 29.1 | 0.11 |

The sizes of the lipid nanocapsules obtained by both methods are generally in very good agreement with an absolute mean deviation of 5.3 nm. Average sizes ranging from 25 to 100 nm, within the desired range, are observed.

Significantly reduced polydispersity indices by a factor of 1.3 to 4.1 compared to the batch process are obtained. Thus, the process according to the invention makes it possible to obtain lower polydispersity indices than for a comparative batch process.

### Change of Scale of the Continuous Process According to the Invention

Comparative tests of the size of the lipid nanocapsules and the polydispersity index (PDI) of the formulation at an SOR ratio of 1.86 (65% by weight of Kolliphor® HS15, 35% by weight of Labrafac® WL1349) were carried out up to a fatty phase flow rate x32 (ie 3.41 mL/min).

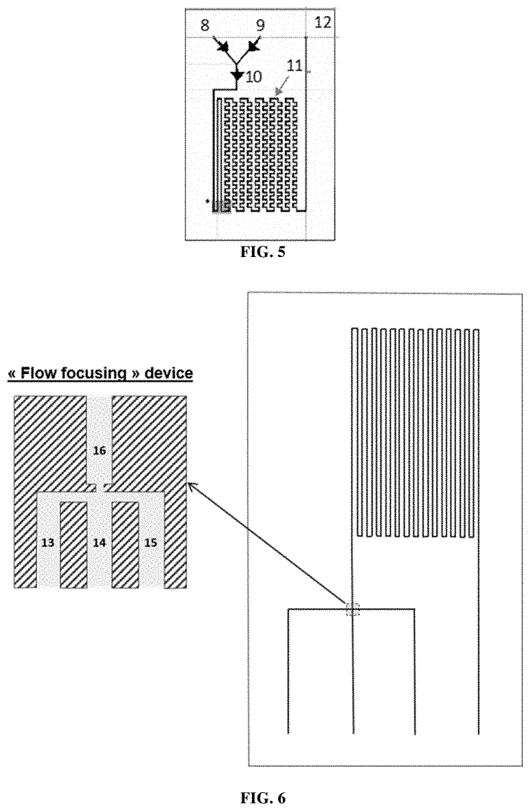

FIG. 5 shows these results.

It is observed that no significant modification of the size of the lipid nanocapsules and of the polydispersity index obtained was observed.

Thus, the process according to the invention is robust and makes it possible to increase the quantity of lipid nanocapsules produced without modifying the characteristics of these lipid nanocapsules.

### Influence of the Nature of the Co-Surfactant and of the Fatty Substance

Table 3 below shows formulations of lipid nanocapsules obtained according to the continuous process of the invention, the fatty phase composition of which consists of Kolliphor® HS 15 (surfactant), Labrafil® M1944 CS (fatty substance) and Transcutol 0 HP (co-surfactant). Comparative tests for four formulations were carried out at a SOWR ratio of 0.047 and at room temperature.

TABLE 3

| Fatty phase composition (% by weight) | | | Continuous CPI | | Batch CPI | |
|---|---|---|---|---|---|---|
| Kolliphor ® | Transcutol ® | Labrafil ® | Size | | Size | |
| HS15 | HP | M1944 CS | (nm) | PDI | (nm) | PDI |
| 40 | 10 | 50 | 35.5 ± 1.9 | 0.06 ± 0.02 | 32.8 ± 1.7 | 0.14 ± 0.01 |
| 50 | 10 | 40 | 27.9 ± 0.5 | 0.12 ± 0.01 | 23.8 ± 1.3 | 0.08 ± 0.01 |
| 50 | 20 | 30 | 24.7 ± 0.7 | 0.08 ± 0.04 | 21.4 ± 0.9 | 0.13 ± 0.02 |
| 65 | 10 | 25 | 19.8 ± 0.1 | 0.05 ± 0.01 | 18.0 ± 0.2 | 0.08 ± 0.02 |

The results show that the microfluidic transposition of the batch process made it possible to obtain lipid nanocapsules having very substantially the same size and polydispersity index characteristics.

### Influence of the SOWR Ratio

Table 4 below shows test results for increasing the SOWR ratio of formulations by continuous CPI process of lipid nanocapsules having the same fatty phase composition as in Table 3.

The increase in the SOWR ratio for these same compositions did not show any change in the characteristics of the lipid nanocapsules (size and polydispersity index).

TABLE 4

| Fatty phase composition (% by weight) | | | | Continuous CPI | |
|---|---|---|---|---|---|
| Kolliphor ® | Transcutol ® | Labrafil ® | SOWR | Size | |
| HS15 | HP | M1944 CS | Ratio | (nm) | PDI |
| 40 | 10 | 50 | 0.047 | 35.5 ± 1.9 | 0.06 ± 0.02 |
| 40 | 10 | 50 | 0.1 | 35.0 ± 2.1 | 0.07 ± 0.01 |
| 40 | 10 | 50 | 0.3 | 36.8 ± 1.7 | 0.12 ± 0.04 |
| 50 | 10 | 40 | 0.047 | 27.9 ± 0.5 | 0.12 ± 0.01 |
| 50 | 10 | 40 | 0.1 | 27.8 | 0.10 |
| 50 | 10 | 40 | 0.3 | 28.0 ± 1.1 | 0.06 ± 0.02 |
| 50 | 20 | 30 | 0.047 | 24.7 ± 0.7 | 0.08 ± 0.04 |
| 50 | 20 | 30 | 0.1 | 24.1 ± 1.1 | 0.06 ± 0.04 |
| 50 | 20 | 30 | 0.3 | 25.5 ± 0.8 | 0.05 ± 0.01 |
| 65 | 10 | 25 | 0.047 | 19.8 ± 0.1 | 0.05 ± 0.01 |
| 65 | 10 | 25 | 0.1 | 20.1 ± 0.1 | 0.07 ± 0.01 |

Thus, it has been shown by means of the above examples that the process according to the invention is robust and easily industrialized. It makes it possible to obtain lipid nanocapsules having a homogeneous and controlled particle size, that is to say with a very low polydispersity (PDI less than 0.15, or very often less than 0.1). Thus, the process according to the invention makes it possible in particular to produce lipid nanocapsules at different scales.

### Example 3

### Formulation of Lipid Nanocapsules by Continuous CPI Process Using a Microfluidic Pilot Unit Coupled to a “T” Type Formulation Chamber

### Reagents and Products Used

Kolliphor® HS 15: PEG 66012-hydroxystearate sold by BASF,
Labrafac® WL 1349: Triglycerides of capric and caprylic acids sold by Gattefosse,
Span® 80: Sorbitan monooleate sold by BASF,
MilliQ ultrapure water: prepared using a WATERS device,
Ethanol 95°: used for cleaning the microfluidic system.

### Specific Material and Conditions

Mettler Toledo weighing scale: for the preparation of the formulation,
Heating/stirring plate IKA C-MAG HS7: for the preparation of the formulation,
Fisherband Polystat 36 thermostatic bath, to maintain the oily phase at temperature and thermoregulate the injection capillaries,
Polyetheretherketone (PEEK) capillary with an internal diameter of 1 mm, for supplying the microfluidic chip with the fatty phase,
Polyetheretherketone (PEEK) capillary with an internal diameter of 1 mm, for supplying the microfluidic chip with the aqueous phase,
Polyetheretherketone (PEEK) capillary with an internal diameter of 1 mm, for the exit of the mixture from the microfluidic chip,
OBI MK3 pressure controller, to inject the fatty phase and the aqueous phase into the microfluidic chip,
Elveflow MFS5 flowmeter, to control the flow rate of the fatty phase,
Bronkhorst Ml 4 flowmeter, to control the flow rate of the aqueous phase,
Microsoft Surface Pro tablet+ESI software, for IT management of flowmeters and pressure controller

The device used in this second example is shown in FIG. 4.

The first microchannel 4 allows the injection of the aqueous phase consisting of MilliQ ultrapure water filtered at 0.2 μm. The second microchannel 5 allows the injection of the fatty phase consisting of a fatty substance, Labrafac® WL 1349, a surfactant, Kolliphor® HS 15 and optionally a co-surfactant, Span® 80. Both microchannels 4 and 5 are arranged in the same plane at 45° to each other and are each connected at one of their ends to the bottom of a bottle. Compressed air overpressure is provided by the OBI MK3 air pressure sensor to allow injection of the oily phase and the aqueous phase. The flow rate of each of the phases is monitored by the flow meters (MFS5 and Ml 4) and the compressed air pressure is adjusted by the pressure controller in order to control the flow rates. Both microchannels 4 and 5 open at their other ends into a formulation chamber 6 where the formation of the lipid nanocapsules takes place. The suspension comprising the lipid nanocapsules is recovered through microchannel 7.

TABLE 5

| | |
|---|---|
| Fatty phase composition (% by weight) | Continuous CPI |

| Kolliphor ® | Labrafac ®WL | Span ® | Size | |
|---|---|---|---|---|
| HS15 | 1349 | 80 | (nm) | PDI |
| 35 | 55 | 10 | 106.2 | 0.12 |
| 65 | 25 | 10 | 25.8 | 0.05 |
| 50 | 40 | 10 | 48.5 | 0.09 |

### Example 4

### Formulation of Nanocapsules by Continuous CPI Process Using a Microfluidic Pilot Unit Coupled to a Microfluidic Chip

### Reagents and Products Used

Kolliphor® HS 15: PEG 660 12-hydroxystearate sold by BASF,
Labrafac® WL 1349: Triglycerides of capric and caprylic acids sold by Gattefosse,
Span® 80: Sorbitan monooleate sold by BASF,
MilliQ ultrapure water: prepared using a WATERS device,
Ethanol 95°: used for cleaning the microfluidic system.

### Specific Material and Conditions

Mettler Toledo weighing scale: for the preparation of the formulation,
Heating/stirring plate IKA C-MAG HS7: for the preparation of the formulation,
Fisherband Polystat 36 thermostatic bath, to maintain the oily phase at temperature and to thermostate the injection capillaries,
Polyetheretherketone (PEEK) capillary with an internal diameter of 1 mm, for supplying the micro fluidic chip with the fatty phase,
Polyetheretherketone (PEEK) capillary with an internal diameter of 1 mm, for supplying the microfluidic chip with the aqueous phase,
Polyetheretherketone (PEEK) capillary with an internal diameter of 1 mm, for the exit of the mixture from the microfluidic chip,
OBI MK3 pressure controller, to inject the fatty phase and the aqueous phase into the microfluidic chip,
Elveflow MFS5 flowmeter, to control the flow rate of the fatty phase,
Bronkhorst Ml 4 flowmeter, to control the flow rate of the aqueous phase,
Microsoft Surface Pro tablet+ESI software, for IT management of flowmeters and pressure controller
Polyetheretherketone (PEEK) microfluidic chip

The device used in this second example is shown in FIG. 5.

The first microchannel 8 allows the injection of the aqueous phase consisting of MilliQ ultrapure water filtered in-line at 0.2 μm. The second microchannel 9 allows the injection of the fatty phase consisting of a fatty substance, Labrafac® WL 1349, a surfactant, Kolliphor® HS 15 and optionally a co-surfactant, Span®80. Both microchannels 8 and 9 are arranged in the same plane at 90° to each other and are each connected at one of their ends to the bottom of a bottle. Compressed air overpressure is provided by the OBI MK3 air pressure sensor to allow injection of the oily phase and the aqueous phase. The flow of each phase is monitored by the flow meters (MFS5 and Ml 4) and the compressed air pressure is adjusted by the pressure controller in order to control the flow rates. Both microchannels open at their other ends into a mixing zone which can be formed as an “accident” in the form of slots 11 where the formation of the lipid nanocapsules takes place. The suspension comprising the lipid nanocapsules is recovered through microchannel 10.

The temperature was set at 50° C., the fatty phase flow rate at 106 μL/min and the SOWR ratio at 0.05.

TABLE 6

| | |
|---|---|
| Fatty phase composition (% by weight) | Continuous CPI |

| Kolliphor ® | Labrafac ®WL | Span ® | Size | |
|---|---|---|---|---|
| HS15 | 1349 | 80 | (nm) | PDI |
| 65 | 25 | 10 | 26.6 | 0.10 |
| 65 | 25 | 10 | 42.3 | 0.09 |
| 35 | 55 | 10 | 82.2 | 0.08 |
| 50 | 40 | 10 | 44.3 | 0.08 |

### Example 5

### Encapsulation of Miltefosine in Lipid Nanocapsules Formulated by Continuous CPI Process Using a “T” Type Formulation Chamber and Syringe Pumps

### Reagents and Products Used

Kolliphor® HS 15: PEG 660 12-hydroxystearate sold by BASF,
Labrafac® WL 1349: Triglycerides of capric and caprylic acids sold by Gattefosse,
Span® 80: Sorbitan monooleate sold by BASF,
MilliQ ultrapure water: prepared using a WATERS device,
Tetrahydrofuran: used for cleaning the microfluidic system.

### Specific Material and Conditions

Mettler Toledo weighing scale: for the preparation of the formulation,
Heating/stirring plate IKA C-MAG HS7: for the preparation of the formulation,
Polyetheretherketone (PEEK) capillary with an internal diameter of 1 mm, for supplying the formulation chamber with the fatty phase,
Polyetheretherketone (PEEK) capillary with an internal diameter of 1 mm, for supplying the formulation chamber with the aqueous phase,
Polyetheretherketone (PEEK) capillary with an internal diameter of 1 mm, for the exit of the mixture from the formulation chamber,
Harvard Apparatus PHD 2000 infusion syringe pump to inject the fatty phase into the formulation chamber,
Harvard Apparatus PHD Ultra (or ISCO 100DX) syringe pump for injecting the aqueous phase into the formulation chamber.

The device used in this second example is shown in FIG. 4.

The first microchannel 4 allows the injection of the aqueous phase consisting of MilliQ ultrapure water filtered at 0.2 μm. The second microchannel 5 allows the injection of the fatty phase consisting of a fatty substance, Labrafac WL 1349, of a pharmacologically active ingredient (anti-infective and anti-cancer), Miltefosine, of a surfactant, Kolliphor® HS15 and optionally a co-surfactant, Span® 80. Miltefosine is initially solubilized in Labrafac WL 1349 for the preparation of the fatty phase. Both microchannels 4 and 5 are arranged in the same plane at 45° to each other and are each connected at one of their ends to a syringe pump allowing the flow rate control of each of the phases. Both microchannels 4 and 5 open at their other ends into a formulation chamber 6 where the formation of the lipid nanocapsules takes place. The suspension comprising the lipid nanocapsules loaded with Miltefosine is recovered via microchannel 7.

### Encapsulation of Miltefosine

Table 7 below shows comparative tests of the results of the formulations of miltefosine lipid nanocapsules obtained by continuous CPI process.

The temperature was set at 37° C., the oil phase flow rate at 425 μL/min and the SOWR ratio at 0.047.

TABLE 7

| | | | | Continuous CPI | | |
|---|---|---|---|---|---|---|
| Fatty phase composition (% by weight) | | | Composition of Miltefosine | | | Zeta |
| Kolliphor ® | Labrafac ®WL | Span ® | solubilized in Labrafac | Size | | Potential |
| HS15 | 1349 | 80 | (% by weight) | (nm) | PDI | (mV) |
| 50 | 40 | 10 | 0 | 42.0 | 0.06 | −3.6 |
| | | | 2.5 | 39.0 | 0.07 | −3.9 |
| | | | 5 | 37.0 | 0.08 | −4.7 |

The sizes of the lipid nanocapsules of encapsulated Miltefosine are on the whole in very good agreement with the formulation of lipid nanocapsules without Miltefosine with an absolute mean deviation comprised of 3.0 and 5.0 nm.

The polydispersity indices are low and not very significantly different between the formulations with or without encapsulated miltefosine.

The Zeta potential (surface charge) decreases not very significantly with increasing miltefosine composition with an absolute mean deviation of 0.3 and 1.1 mV.

Thus, the results show that the continuous CPI process allowed the formulation at low temperature (37° C.) of lipid nanocapsules loaded with a pharmacologically active ingredient, having substantially the same characteristics of size, polydispersity index and Zeta potential as the uncharged nanocapsules.

### Influence of the SOWR Ratio

Table 8 below shows the results of tests of increasing the SOWR ratio of formulations by continuous CPI process of lipid nanocapsules loaded with Miltefosine having the same fatty phase composition as in Table 7.

The increase in the SOWR ratio for these same compositions did not show any change in the characteristics of the lipid nanocapsules (size and polydispersity index).

TABLE 8

| Fatty phase composition (% by weight) | | | Composition of Miltefosine | | Continuous CPI | |
|---|---|---|---|---|---|---|
| Kolliphor ® | Labrafac ®WL | Span ® | solubilized in Labrafac | SOWR | Size | |
| HS15 | 1349 | 80 | (% by weight) | Ratio | (nm) | PDI |
| 50 | 40 | 10 | 5 | 0.047 | 42.0 | 0.06 |
| | | | | 0.200 | 39.0 | 0.04 |

Thus, it has been shown by means of the above examples that the process according to the invention is robust and can be easily industrialized. It allows the formulation at low temperature (37° C.) of lipid nanocapsules loaded with a pharmacologically active ingredient, in particular an anti-cancer agent and an anti-infectious agent (Miltefosine). The lipid nanocapsules loaded with pharmacologically active ingredient have a homogeneous and controlled particle size, that is to say with a very low polydispersity of less than 0.1. Thus, the process according to the invention makes it possible in particular to produce lipid nanocapsules at different scales.

1-15. (canceled)

16. A continuous process for nano-emulsification, wherein said process is carried out by concentration phase inversion (CPI) in a microfluidic reactor, and comprising the following steps:
(a) injection into a first microchannel of an aqueous phase, said first microchannel opening into a formulation chamber,
(b) injection into a second microchannel of a fatty phase comprising one or more fatty substances, and one or more surfactants, said second microchannel opening into said formulation chamber, then
(c) mixing of the aqueous phase and the fatty phase in said formulation chamber, then
(d) recovery, at the outlet of the formulation chamber, of a suspension comprising lipid nanocapsules.

17. The process according to claim 16, wherein the surfactant(s) are chosen from nonionic hydrophilic surfactants, and mixtures thereof.

18. The process according to claim 17, wherein the surfactant(s) are chosen from mono- and di-esters of fatty acid and of polyethylene glycol, and mixtures thereof.

19. The process according to claim 18, wherein the surfactant(s) are chosen from mono- and di-esters of stearic acid and of polyethylene glycol, and mixtures thereof.

20. The process according to claim 16, wherein the fatty substance(s) are chosen from glycerol mono-esters, di-esters and tri-esters, polyethylene glycol mono-esters and di-esters, and mixtures thereof.

21. The process according to claim 20, wherein the fatty substance(s) are chosen from C₈-C₁₈ triglycerides, and mixtures thereof.

22. The process according to claim 21, wherein the fatty substance(s) are chosen from capric and caprylic acid triglycerides and mixtures thereof.

23. The process according to claim 16, wherein the fatty phase further comprises one or more co-surfactants.

24. The process according to claim 23, wherein the fatty phase further comprises one or more co-surfactants, chosen from nonionic surfactants.

25. The process according to claim 24, wherein the fatty phase further comprises one or more co-surfactants, chosen from sorbitan monooleate or diethylene glycol mono-ethyl ether and mixtures thereof.

26. The process according to claim 16, wherein the weight ratio of the sum of the flow rates of surfactants and co-surfactants to the flow rate of fatty substances in the formulation chamber is between 0.8 and 4.

27. The process according to claim 16, wherein the weight ratio of the sum of the flow rates of surfactant, co-surfactants and fatty substances to the flow rate of the aqueous phase in the formulation chamber is between 0.03 and 0.3.

28. The process according to claim 16, wherein the fatty phase further comprises water, in a content of between 0% and 30% by weight, relative to the total weight of the fatty phase.

29. The process according to claim 16, wherein the first microchannel is thermalized at a temperature between 20° C. and 70° C.

30. The process according to claim 16, wherein the second microchannel is thermalized at a temperature between 20° C. and 70° C.

31. Lipid nanocapsules obtained by the process according to claim 23, wherein they comprise one or more co-surfactants chosen from nonionic surfactants.

32. The lipid nanocapsules according to claim 31, wherein they further comprise a pharmacologically active ingredient.

33. The lipid nanocapsules according to claim 31, wherein they have a particle size of between 15 and 120 nm.

34. The lipid nanocapsules according to claim 31, wherein they have a polydispersity index of between 0.05 and 0.2.

35. A method of treating a subject in need thereof, said method comprising administering to said subject a therapeutically effective amount of at least one lipid nanocapsule according to claim 31.
